(19) United States
(12) Patent Application Publication
(10) Pub. No.: US 2024/0091472 A1
(43) Pub. Date: Mar. 21, 2024

(54) MANUAL RESPIRATORY RESUSCITATION SYSTEM

(71) Applicant: University of Pittsburgh - Of the Commonwealth System of Higher Education, Pittsburgh, PA (US)

(72) Inventor: Travis Lux Fernald, Pittsburgh, PA (US)

(21) Appl. No.: 17/768,055

(22) PCT Filed: Oct. 13, 2020
(86) PCT No.: PCT/US2020/055353

§ 371 Date: Apr. 11, 2022

Related U.S. Application Data
(60) Provisional application No. 62/914,780, filed on Oct. 14, 2019.

(51) Int. Cl. A61M 16/00; A61M 16/06

(52) U.S. Cl. CPC A61M 16/0084; A61M 16/0616

(57) ABSTRACT

A device for manual respiratory resuscitation or ventilation assistance includes a mask suitable to form a seal with a patients face, a compressible bag, and an extending, flexible conduit connected to a first opening in the bag and to a first opening in the mask to place the mask in fluid connection with the bag. The extending, flexible conduit is of sufficient length to allow a user to use one or more portions of the user's body other than either of the user's hands to compress the compressible bag.

### CROSS-REFERENCE TO RELATED APPLICATIONS

This application claims benefit of U.S. Provisional Patent Application Ser. No. 62/914,780, filed Oct. 14, 2019, the disclosure of which is incorporated herein by reference.

### BACKGROUND

The following information is provided to assist the reader in understanding technologies disclosed below and the environment in which such technologies may typically be used. The terms used herein are not intended to be limited to any particular narrow interpretation unless clearly stated otherwise in this document. References set forth herein may facilitate understanding of the technologies or the background thereof. The disclosure of all references cited herein are incorporated by reference.

Manual respiratory resuscitation or manual ventilation assistance devices, bags or respiratory bag-valve masks as illustrated in FIGS. 1A and 1B are, for example, used for resuscitation and manual ventilation. A bag valve mask typically includes a flexible, compressible or collapsible air chamber or bag which is attached to a face mask via, for example, a short, rigid conduit and a valve mechanism/system (for example, including a one-way inhalation valve, a one-way exhalation valve, a one-way bag refill valve, an overpressure valve, an entrainment valve etc.). When the face mask is properly placed in sealing connection with a patient's face/mouth, the bag is squeezed/compressed to a compressed (lower internal volume) state, and the device forces air into the patient's lungs. When the bag is released to a resting or expanded (greater internal volume) state, it self-inflates (for example, from the end thereof opposite the face mask), with either ambient air or a low pressure oxygen flow (which may be supplied by a compressed oxygen source such as a regulated cylinder). During self-inflation or expansion, the patient's lungs deflate as the patient's lungs exhale to the ambient environment. A one-way valve may prevent exhalation/backflow into the bag. A bag-valve mask may be used without being attached to an oxygen tank to provide ambient air to the patient. A bag-valve mask may also be connected to a separate reservoir bag which can be filled with oxygen or breathing gas including oxygen from a compressed/pressurized oxygen/breathing gas source. Use of the device in connection with an oxygen/breathing gas source can increase the amount of oxygen delivered to the patient to nearly 100%. The construction and operation of representative, currently available manual resuscitation devices or bags are, for example, disclosed in US Patent Application Publication No. 2017/0157348 and Korean Patent No. KR101730091B1, the disclosures of which are incorporated herein by reference.

Although possible, it is often quite difficult for a single person to operate a bag-valve mask. Using one hand to squeeze the bag may, for example, cause rapid fatigue. Moreover, it is very difficult to secure/seal the mask to the patient's face with a single hand. Therefore, it is strongly recommended that two users/rescuers operate a bag-valve mask. One rescuer holds the mask to the patient's face with both hands as illustrated in FIG. 1B and focuses entirely on maintaining a leak-proof mask seal. The other rescuer squeezes the bag and focuses entirely on breath (or tidal volume) and timing.

### SUMMARY

In one aspect, a device for manual respiratory resuscitation or ventilation assistance includes a mask suitable to form a seal with a patient's face, a compressible bag, and an extending, flexible conduit connected to a first opening in the bag and to a first opening in the mask to place the mask in fluid connection with the bag. The extending, flexible conduit is of sufficient length to allow a user to use one or more portions of the user's body other than either of the user's hands to compress the compressible bag. In a number of embodiment, the bag is self-inflating (that is, the bag has shape memory to re-inflate to a resting, expanded or non-compressed state after removal of compressive force). The extending, flexible conduit may, for example, be of sufficient length to allow the user to compress the bag between the user's arm and the single user's side. In a number of embodiments, the extending, flexible conduit is at least 12 inches in length, at least 18 inches in length, at least 24 inches in length, at least 30 inches in length, at least 36 inches in length, at least 42 inches in length, or longer, thereby providing separation between the compressible bag and the mask to facilitate use of one or more portions of the user's body other than either of the user's hands to compress/relax the compressible bag in, for example, a periodic or timed manner.

The device may, for example, further include a reservoir bag in fluid connection with the compressible bag and the mask to deliver oxygen to the patient. The second opening may, for example, be formed in the compressible bag. In a number of embodiments, the compressible bag is contoured to facilitate compression thereof using a portion of the user's body other than either of the user's hands. The compressible bag may, for example, include at least one indentation in a side thereof into which the user's arm (or side) can be placed.

In another aspect, a method of manually applying respiratory resuscitation or ventilation assistance to a patient includes providing a device including a mask suitable to form a seal with a patient's face, a compressible bag, and an extending, flexible conduit connected to a first opening in the bag and to a first opening in the mask to place the mask in fluid connection with the bag, placing the mask in sealing connection with the patient's face, extending the extending flexible conduit to enable contact of one or more portions of a user's body other than either of the user's hands, and compressing the compressible bag using the one or more portions of the user's body other than either of the user's hands. In a number of embodiments, both hands of the user are used to maintain the mask in sealing connection with the patient's face during operation of the device. In a number of embodiments, the bag is self-inflating. The bag may, for example, be repeatedly compressed by the user (allowing the bag to self-inflate between compressions).

In a number of embodiment, the extending, flexible conduit is at least 12 inches in length, at least 18 inches in length, at least 24, inches in length, at least 30 inches in length, at least 36 inches in length, at least 42 inches in length, or longer, thereby providing for separation between the compressible bag and the mask as discussed above.

The device may, for example, further include a reservoir bag in fluid connection with the compressible bag and the mask to deliver oxygen (or a breathing gas including oxygen). In a number of embodiments, the compressible bag is contoured to facilitate compression thereof using a portion of the user's body other than either of the user's hands. The compressible bag may, for example, include at least one indentation in a side thereof into which the user's arm (or a user's side) can be placed.

In still a further aspect, a method of operating a device for manual respiratory resuscitation or manual ventilation assistance includes placing a mask of the device which includes a seal member to form a seal with the face of a patient in (sealing) connection with the patient's face, extending a flexible conduit connected to a first opening in a compressible bag and to a first opening in the mask to one or more portions of the user's body other than either of the user's hands to compress the compressible bag using the one or more portions of the user's body.

The present devices, systems, and methods, along with the attributes and attendant advantages thereof, will best be appreciated and understood in view of the following detailed description taken in conjunction with the accompanying drawings.

### BRIEF DESCRIPTION OF THE DRAWINGS

FIG. 1A illustrates a representative embodiment of a currently available resuscitation device, bag or respiratory bag-valve mask.

FIG. 1B illustrates use of a currently available resuscitation device, bag or respiratory bag-valve mask on a patient by two persons.

FIG. 2A illustrates a perspective view of a representative embodiment of a resuscitation or ventilation assistance device or system hereof.

FIG. 2B illustrates schematically the device or system of FIG. 2A including a reservoir bag.

FIG. 3 illustrates a top perspective view of a mask of the resuscitation system of FIG. 2A.

FIG. 4A illustrates a partially hidden line, perspective view of a compressible or squeezable bag of the resuscitation system of FIG. 2A.

FIG. 4B illustrates another partially hidden line, perspective view of the compressible bag of FIG. 4A.

FIG. 5 illustrates a perspective view of the flexible conduit or tube of the resuscitation device or system of FIG. 2A.

FIG. 6A illustrates a side view of another embodiment of a compressible bag for use in the resuscitation system hereof including loops to attach a strap.

FIG. 6B illustrates another side view of the compressible bag of FIG. 6A, wherein the compressible bag is rotated approximately 90° around the axis A of the compressible bag from the view of FIG. 6A.

### DESCRIPTION

The present devices, systems, and methods, along with the attributes and attendant advantages thereof, will best be appreciated and understood in view of the following description taken in conjunction with any accompanying drawings.

It will be readily understood that the components of the embodiments, as generally described and illustrated in the figures herein, may be arranged and designed in a wide variety of different configurations in addition to the described example embodiments. Thus, the following more detailed description of the example embodiments, as represented in the figures, is not intended to limit the scope of the embodiments, as claimed, but is merely representative of example embodiments.

Reference throughout this specification to “one embodiment” or “an embodiment” (or the like) means that a particular feature, structure, or characteristic described in connection with the embodiment is included in at least one embodiment. Thus, the appearance of the phrases “in one embodiment” or “in an embodiment” or the like in various places throughout this specification are not necessarily all referring to the same embodiment.

Furthermore, described features, structures, or characteristics may be combined in any suitable manner in one or more embodiments. In the following description, numerous specific details are provided to give a thorough understanding of embodiments. One skilled in the relevant art will recognize, however, that the various embodiments can be practiced without one or more of the specific details, or with other methods, components, materials, et cetera. In other instances, well known structures, materials, or operations are not shown or described in detail to avoid obfuscation.

As used herein and in the appended claims, the singular forms “a,” “an”, and “the” include plural references unless the context clearly dictates otherwise. Thus, for example, reference to “a bag” includes a plurality of such bags and equivalents thereof known to those skilled in the art, and so forth, and reference to “the bag” is a reference to one or more such bags and equivalents thereof known to those skilled in the art, and so forth. Recitation of ranges of values herein are merely intended to serve as a shorthand method of referring individually to each separate value falling within the range. Unless otherwise indicated herein, and each separate value as well as intermediate ranges are incorporated into the specification as if it were individually recited herein. All methods described herein can be performed in any suitable order unless otherwise indicated herein or otherwise clearly contraindicated by the text.

In a number of embodiments, devices, systems, and methods hereof provide a single-user solution for bag-valve masks used in resuscitation. In a number of embodiments, a manual resuscitation device hereof (for use, in manual resuscitation in, for example, CPR) includes an extending, flexible conduit between a compressible bag or bladder and a mask which forms a seal with a patient's face. In general, the extending, flexible conduit is of a sufficient length to allow a single person or rescuer to use both hands in connection with the mask/patient while the person/rescuer uses another part or parts of their body to compress/release the compressible bag or bladder. The compressible bag may, for example, be compressed between two parts of the user's body or between a part of the user's body and a surface other than the body of the patient. The compressible bag may, for example, be held and pumped between the operator's side and arm, similar to manner in which a bag of a bagpipe is operated. The compressible bag or bladder may, for example, be contoured so that it readily fits between an operator's or rescuer's arm and side or for another mode of operation hereunder. The compressible bag may, for example, alternatively be compressed between the user's knee or foot and the ground.

A mode or manner of operation wherein an operator, user or rescuer operates the compressible bag or bladder with a part of parts of their body other than the hands allows use of the device by a single operator, freeing both the operator's hands to, for example, hold the mask on the patient's face and to tilt the patient's chin upward to open the patient's airway. The arm/side pumping style is less fatiguing than using a hand or hands to pump the bag, allowing an operator to operate the device longer that previously available devices without having to, for example, switch off with another operator. The extending, flexible conduit between the compressible, self-inflating bag or bladder and the mask of the devices hereof may, for example, extend to a sufficient length or distance that an operator's thighs or even the operator's foot may be used to compress the bag. As described above, use of larger muscle groups (rather than solely the hands) results in less fatigue and frees the hands of the user.

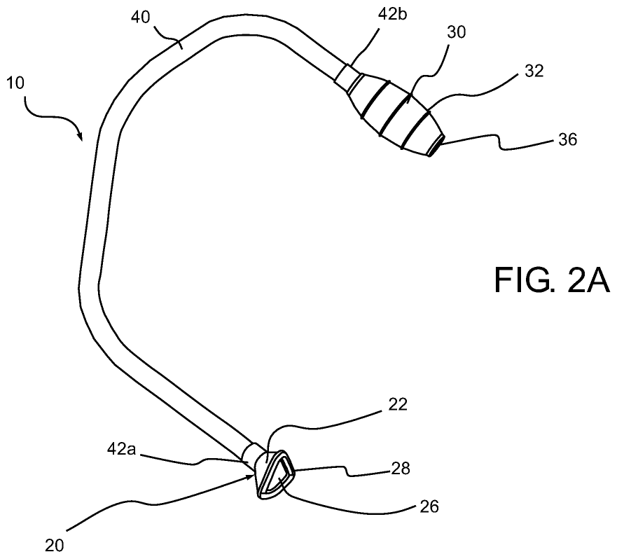

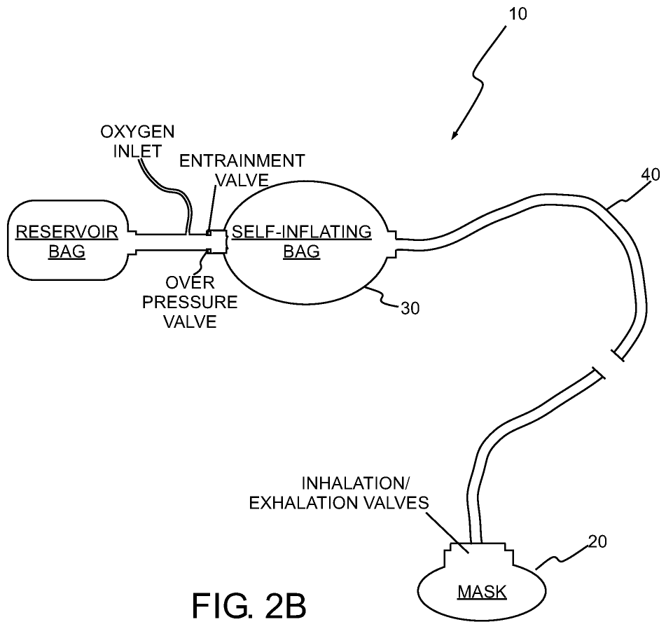

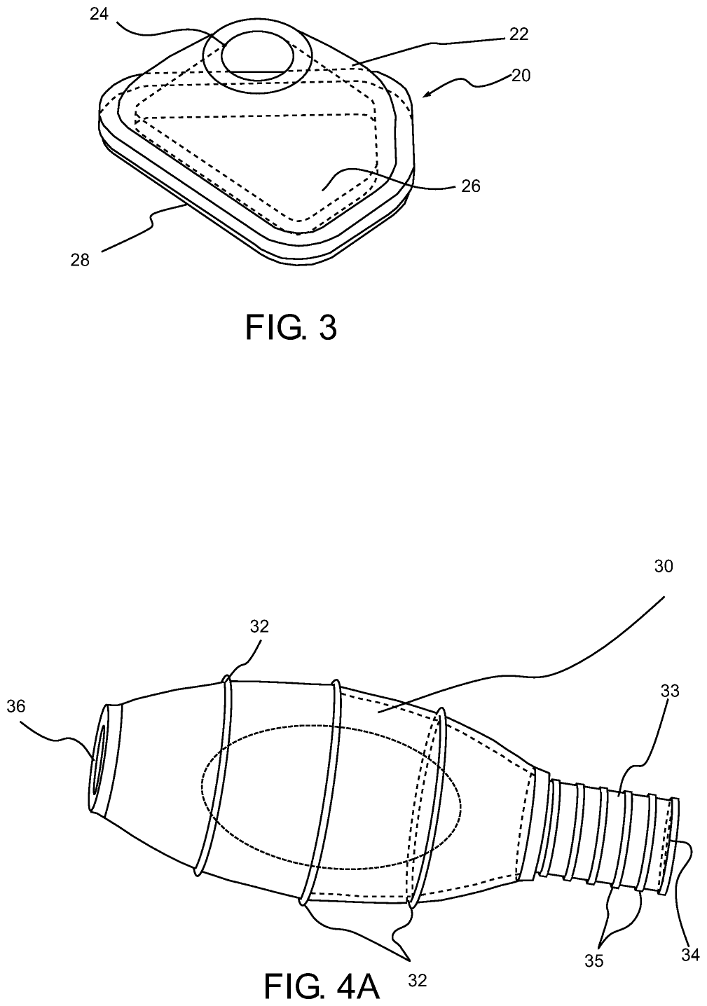

FIGS. 2A and 2B illustrates a device or system 10 hereof and the components thereof. Mask 20 of system 10 includes a generally rigid shell 22 having a first opening 24 (see, for example, FIG. 3) on a first end thereof for connection to extending, flexible conduit 40 via a first connector 42a thereof and a second opening 26 to be operatively connected to a patient's face. A seal or sealing cushion 28 (for example, formed from a silicone rubber or other flexible sealing material) may be connected/sealed to a perimeter of second opening 26 to conform to and form a seal with the patient's face around the patient's nose and mouth (see FIGS. 2A and 3). As illustrated in FIG. 2B, device or system 10 can further include a reservoir bag for delivery of oxygen (or other breathing gas including oxygen) in fluid connection with mask 20 as described above.

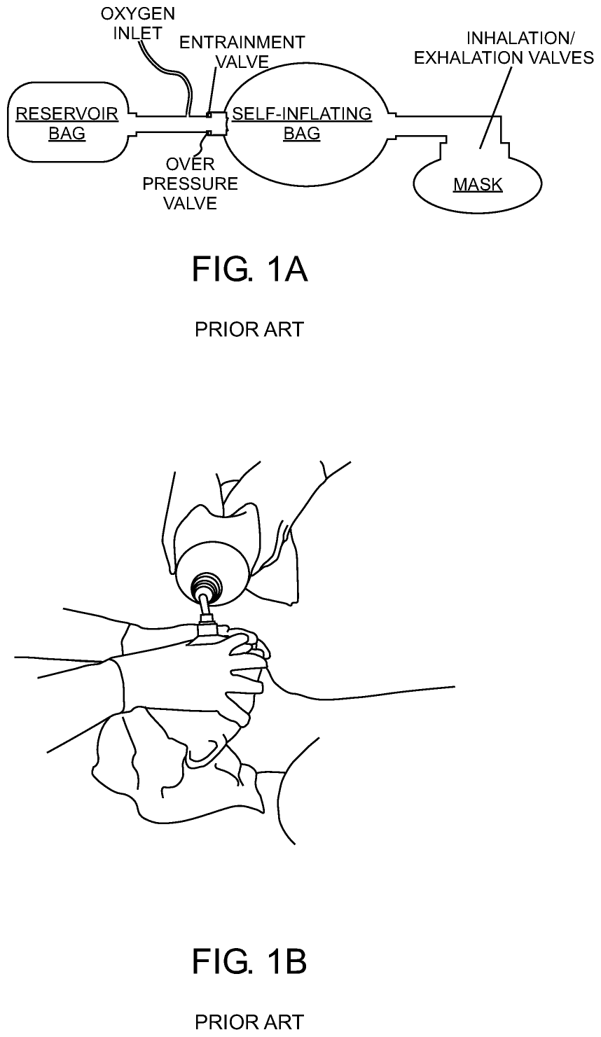

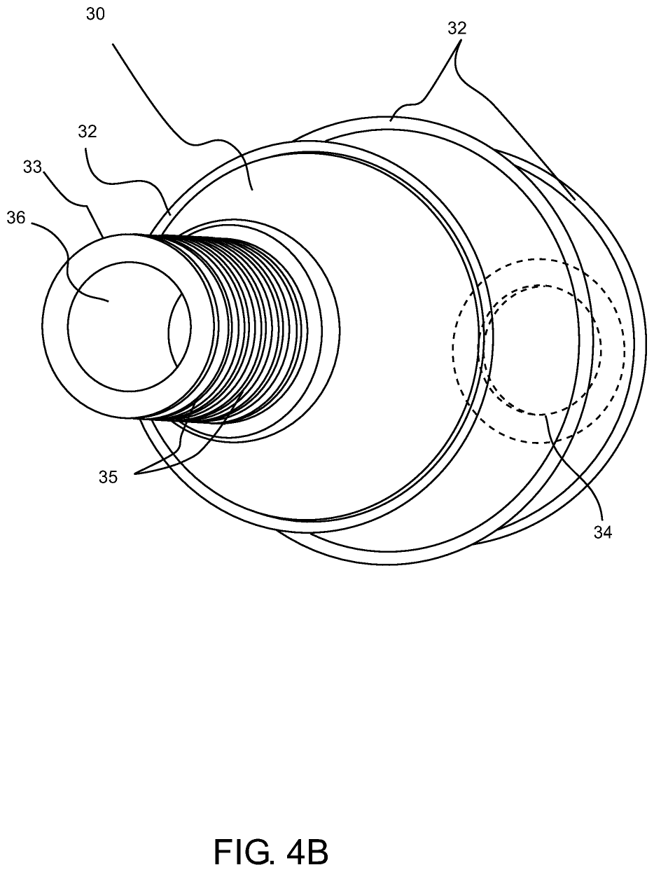

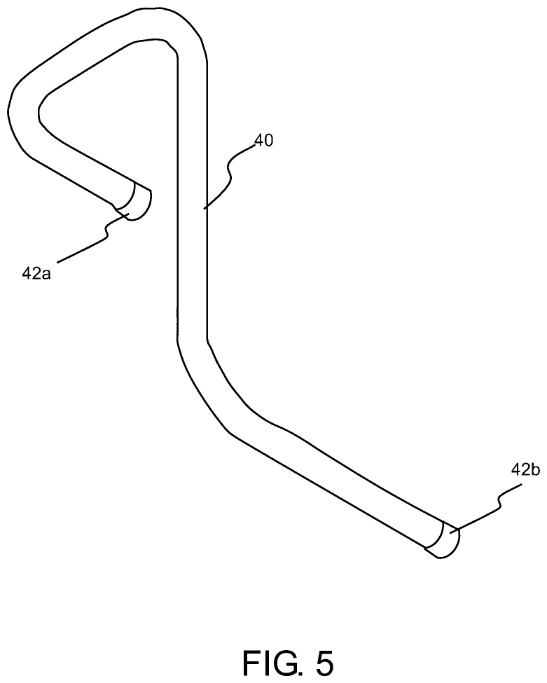

As known in the art, bag or bladder 30 of system 10 hereof is compressible and may have shape memory such that, when not compressed, the bag naturally springs back to its fully expanded, resting, or non-compressed state, which is shown in the figures. The bag in the device illustrated in FIG. 1B is illustrated in a compressed state. As illustrated, for example, in FIGS. 4A and 4B, such shape memory may, for example, be facilitated by ribs 32 of bag 30. Bag 30 includes a first opening 34 formed in a connector section 33 (which may be ribbed with ribs 35; see FIG. 4A) to form a sealed connection with a second connector 42b of extending, flexible conduit 40 (see FIGS. 2A and 5). A second opening 36 may, for example, be provided on an opposite end of bag 30. Second opening 36 may, for example, be used to connect to an oxygen reservoir bag etc. as illustrated in FIG. 2B. As also described above, bag 30 may be contoured in a number of embodiments hereof to, for example, be placed between an operator's side and arm for operation in the manner of a bag of a bagpipe (or to conform to one or more other parts of the user's body). Bag 30 may, for example, have semicircular, oval or otherwise shaped areas or indentations in the sides (represented by the area within broken lines in FIG. 4A) into which the arm and side can fit snuggly. Alternatively bag 30 may be fabricated or contoured in the form of bellows.

The mask may, for example, be provided with connectors such as loops or slots (not shown) to connect to one or more straps to the mask to assist in sealing the mask to the patient's face. Such strap may, however, be undesirable in some situations in which relatively rapid removal of the mask is desirable in the case of emesis/vomiting.

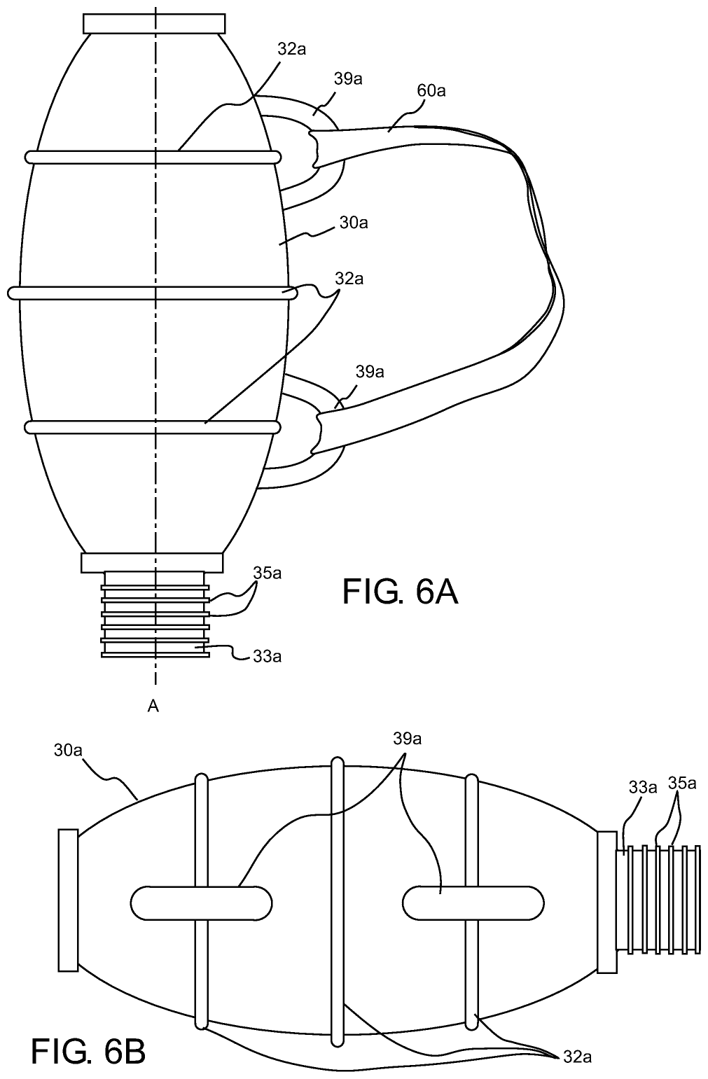

FIGS. 6A and 6B illustrate another embodiment of a collapsible or compressible bag 30a hereof. Elements of bag 30a are labeled similarly to corresponding elements of mask 30 with the addition designation “a” thereto. Loops 39a on bag 30a may, for example, be used to attach a strap 60a to bag 30a which may, for example, be used to stabilize or hold bag 30a over or upon a portion of the user's body to facilitate compression/expansion. Strap 60 may, for example, hold bag 30a to be positioned on the inside of the user's arm to facilitate compression/expansion between the user's arm and the user's side. Strap 60a may, for example, be formed from an elastic material or may be adjustable (for example, via a buckle-like mechanism as known in the belt/strap arts—not shown) to stabilize bag 30a upon the user.

In general, extending, flexible conduit 40 of system 10 is of sufficient flexibility and length to be readily extended to the part(s), portion(s) and/or appendage(s) of the operator's body (other than the user's hands) to be used in compressing bag 30. For example, extending, flexible conduit 40 may extend to a sufficient length to be operated in the manner of a bagpipe bag between the operator's side and arm. Alternatively, extending, flexible conduit 40 may extend to a sufficient length to be operated in connection with the operator's thigh(s), knee(s) or foot/feet. In a number of embodiments, extending, flexible conduit 40 is at least 12 inches in length, at least 18 inches in length, at least 24 inches, at least 30 inches in length, at least 36 inches in length, at least 42 inches in length, or longer. Extending, flexible conduit 40 may, for example, be fabricated from silicone, polyvinyl siloxane (PVS), or a similar material. Virtually any manual, compressible bag resuscitation/ventilation device or system (regardless of the valving system or other characteristics thereof) can be readily redesigned to include the extending, flexible conduit hereof.

The foregoing description and accompanying drawings set forth a number of representative embodiments at the present time. Various modifications, additions and alternative designs will, of course, become apparent to those skilled in the art in light of the foregoing teachings without departing from the scope hereof, which is indicated by the following claims rather than by the foregoing description. All changes and variations that fall within the meaning and range of equivalency of the claims are to be embraced within their scope.

1. A device for manual respiratory resuscitation or ventilation assistance, comprising: a mask suitable to form a seal with a patient's face, a compressible bag and an extending, flexible conduit connected to a first opening in the compressible bag and to a first opening in the mask to place the mask in fluid connection with the compressible bag, the extending, flexible conduit being of sufficient length to allow a user to use one or more portions of the user's body other than either of the user's hands to compress the compressible bag.

2. The device of claim 1 wherein the compressible bag is self-inflating.

3. The device of claim 2 wherein the extending, flexible conduit is of sufficient length to allow the user to compress the compressible bag between the user's arm and the single user's side.

4. The device of claim 2 wherein the extending, flexible conduit is at least 18 inches in length.

5. The device of claim 2 wherein the extending, flexible conduit is at least 24 inches in length.

6. The device of claim 2 wherein the extending, flexible conduit is at least 30 inches in length.

7. The device of claim 1 further comprising a reservoir bag in fluid connection with the compressible bag and the mask to deliver oxygen.

8. The device of claim 1 wherein the compressible bag is contoured to facilitate compression thereof using a portion of the user's body other than either of the user's hands.

9. The device of claim 8 wherein the compressible bag comprises at least one indentation in a side thereof into which the user's arm can be placed.

10. A method of manually applying respiratory resuscitation or ventilation assistance to a patient, comprising:
providing a device including a mask suitable to form a seal with the patient's face, a compressible bag and an extending, flexible conduit connected to a first opening in the compressible bag and to a first opening in the mask to place the mask in fluid connection with the compressible bag;
placing the mask in sealing connection with the patient's face;
extending the extending flexible conduit to enable contact of one or more portions of a user's body other than either of the user's hands; and
compressing the compressible bag using the one or more portions of the user's body other than either of the user's hands.

11. The method of claim 10 wherein both hands of the user are used to maintain the mask in sealing connection with the patient's face.

12. The method of claim 11 wherein the compressible bag is self-inflating.

13. The method of claim 12 wherein the compressible bag is repeatedly compressed by the user.

14. The method of claim 11 wherein the extending, flexible conduit is at least 18 inches in length.

15. The method of claim 11 wherein the extending, flexible conduit is at least 24 inches in length.

16. The method of claim 11 wherein the extending, flexible conduit is at least 30 inches in length.

17. The method of claim 10 further comprising a reservoir bag in fluid connection the compressible bag and the mask to deliver oxygen.

18. The method of claim 10 wherein the compressible bag is contoured to facilitate compression thereof using a portion of the user's body other than either of the user's hands.

19. The method of claim 18 wherein the compressible bag comprises at least one indentation in a side thereof into which the user's arm can be placed.

20. A method of operating a device for manual respiratory resuscitation or ventilation assistance, comprising: placing a mask of the device which includes a seal member to form a seal with the face of a patient in connection with the patient's face, extending a flexible conduit connected to a first opening in a compressible bag and to a first opening in the mask to one or more portions of the user's body other than either of the user's hands to compress the compressible bag, and using the one or more portions of the user's body other than either of the user's hands to compress the compressible bag.
